(19) United States
(12) Patent Application Publication
(10) Pub. No.: US 2013/0089529 A1
(43) Pub. Date: Apr. 11, 2013

(54) AGENT FOR OSTEOGENESIS USING SUBSTANCE-P AND PREPARATION PROCESS THEREOF

(71) Applicants: Young Sook Son, Seoul (KR); Eun Ah Lee, Seoul (KR); Hyun Sook Hong, Seoul (KR)

(72) Inventors: Young Sook Son, Seoul (KR); Eun Ah Lee, Seoul (KR); Hyun Sook Hong, Seoul (KR)

(73) Assignee: UNIVERSITY-INDUSTRY COOPERATION GROUP OF KYUNGHEE UNIVERSITY, Yongin-city, Gyeonggi-do (KR)

(21) Appl. No.: 13/703,299

(22) PCT Filed: Jun. 11, 2010
(86) PCT No.: PCT/KR10/03784

§ 371 Date: Dec. 10, 2012

(51) Int. Cl. A61K 35/28

(52) U.S. Cl. CPC A61K 35/28
USPC 424/93.7

(57) ABSTRACT

The present invention relates to an agent for osteogenesis containing as an active ingredient mesenchymal stem cells (MSCs) that have been mobilized from bone marrow into blood by administration of Substance-P; and a process of preparing the agent.

### TECHNICAL FIELD

The present invention relates to an agent for osteogenesis containing as an active ingredient mesenchymal stem cells (MSCs) that have been mobilized from bone marrow into blood by administration of Substance-P; and a process of preparing the agent.

### BACKGROUND ART

Substance-P is a neuropeptide consisting of 11 amino acids that is expressed in sensory neurons, macrophages, eosinophils, endothelial cells, epithelial cells, and corneal cells such as keratocytes as well as granulation tissue. Several reports have suggested implications of Substance-P in neuro-immune communication on hematopoietic modulation. Bone marrow stroma is innervated by Substance-P nerve fibers, and Substance-P stimulates bone marrow stromal cells through their surface receptor NK-1 to produce stem cell factors and interleukin-1, which may be favorable for hematopoietic stimulation as feeders.

Mesenchymal stem cells (MSCs) of bone marrow have the potential to differentiate into bone or cartilage and are used in a study of diseases that need osteogenesis. According to the conventional methods, MSCs were isolated directly from bone marrow of a patient, which is a complicated procedure. Besides, such methods cannot be frequently performed and are painful procedures for a patient. In a normal physiological state without any wound, MSCs are detected in tissues such as fat tissues and pterygium except bone marrow, but barely in peripheral blood.

Korean Laid-open Publication No. 10-2006-0037176, which is an earlier application by the present inventors, disclosed that Substance-P can mobilize or proliferate MSCs from bone marrow, and provided a wound-healing agent containing as an active ingredient Substance-P and/or MSCs that have been mobilized from bone marrow by treatment of Substance-P.

However, no report has yet stated that the MSCs which have been mobilized from bone marrow into blood by administration of Substance-P can be isolated and used as an active ingredient for an agent for osteogenesis.

### DISCLOSURE OF INVENTION

Technical Problem

The present inventors have performed continuous study on a method to isolate MSCs from bone marrow easily and conveniently without causing pain to a patient, which method can substitute the conventional methods of isolating MSCs directly from bone marrow of a patient. As a result, they discovered that MSCs of bone marrow are mobilized into blood after administration of Substance-P and can be easily isolated from blood and that an agent for osteogenesis containing said isolated MSCs as an active ingredient exhibits an outstanding effect in osteogenesis or bone repair, and completed the present invention.

Solution to Problem

Thus, the first object of the present invention is to provide an agent for osteogenesis containing as an active ingredient MSCs that have been mobilized from bone marrow into blood by administration of Substance-P.

The second object of the present invention is to provide a process of preparing an agent for osteogenesis, comprising isolating MSCs that have been mobilized from bone marrow into blood by administration of Substance-P.

Advantageous Effects of Invention

The agent for osteogenesis containing as an active ingredient MSCs that have been mobilized from bone marrow into blood by administration of Substance-P and the process of preparing the same according to the present invention can easily and conveniently isolate MSCs of bone marrow without causing pain to a patient, contrary to the conventional methods of isolating MSCs directly from bone marrow of a patient. In addition, since the agent exhibits the capacity for osteogenesis in a degree equivalent to general MSCs that have been isolated directly from bone marrow, the present invention for the first time proved that osteogenesis or bone repair is possible by cells isolated from blood.

### BRIEF DESCRIPTION OF DRAWINGS

FIG. 1 is a picture showing osteogenesis of the transplant isolated from the nude mouse 10 weeks after transplantation of MSCs that had been isolated from blood after intravenous injection of Substance-P into a rabbit (pink color: collagen [bone matrix], A: fat tissues, H: bone marrow tissues, dark purple color: fibrous tissues).

FIG. 2 is a high-resolution picture of FIG. 1.

FIG. 3 is a picture showing the staining result of the transplant after treatment with antibodies that respond to rabbit collagen, wherein said transplant was isolated from the nude mouse 10 weeks after transplantation of MSCs that had been isolated from blood after intravenous injection of Substance-P into a rabbit.

FIG. 4 is a picture showing the staining result of normal skin tissues of the mouse after treatment with antibodies that respond to rabbit collagen (blue: collagen, red: cytoplasm).

FIG. 5 is a picture showing osteogenesis of the transplant isolated from the nude mouse 10 weeks after transplantation of MSCs that had been isolated directly from rabbit bone marrow (pink color: collagen [bone matrix], light purple color: residual HA-TCP).

### MODE FOR THE INVENTION

The first aspect of the present invention relates to an agent for osteogenesis containing as an active ingredient MSCs that have been mobilized from bone marrow into blood by administration of Substance-P.

The second aspect of the present invention relates to a process of preparing an agent for osteogenesis, comprising isolating MSCs that have been mobilized from bone marrow into blood by administration of Substance-P.

In the previous study (Korean Laid-open Publication No. 10-2006-0037176), the present inventors discovered that when Substance-P was intravenously injected into a test animal even without wound (e.g., corneal burn), many CD29+ MSCs were mobilized into peripheral blood. They also discovered that in in vitro 3-D collagen gel, Substance-P stimulated migration of MSCs by inducing matrix-degrading enzymes and inhibiting their inhibitors, which explains the mechanism of MSCs' migration from bone marrow.

Specifically, in order to determine a systemic effect of Substance-P separately from the other factors that are found in the wound microenvironment, Substance-P was intravenously injected into the mice without wound, and the mobilization of CD29+ MSCs into peripheral blood was examined. As a result, it was observed that approximately 15 times more CD29+ MSCs were mobilized into peripheral blood in the mice which were intravenously injected with Substance-P, than in the non-injected mice. From the results, the present inventors concluded that Substance-P is expressed at the early stage of the wound-healing process and plays a role in mobilizing MSCs from bone marrow into blood, ultimately to supply MSCs to the corneal wound site and to facilitate corneal repair.

In the subsequent study, the present inventors isolated the MSCs which have been mobilized from bone marrow into blood by administration of Substance-P and transplanted them subcutaneously on the back of a nude mouse. As a result, they confirmed that bone matrix, which is composed of collagen, had indeed been generated. In other words, the present invention for the first time proved that osteogenesis or bone repair is possible by MSCs that have been isolated from blood.

In the present invention, in order to mobilize MSCs from bone marrow into blood, Substance-P can be administered preferably by intravenous injection, subcutaneous injection, endodermis injection or muscular injection, most preferably by intravenous injection.

In the present invention, the effective dosage of Substance-P to mobilize MSCs from bone marrow into blood is 0.1 to 1000 μg/kg, preferably 0.1 to 100 μg/kg. However, the present invention is not limited to said dosage.

In addition, according to the present invention, MSCs are mobilized from bone marrow into blood from the 2nd day to the 4th day, preferably on the 3rd day, after administration of Substance-P. Thus, it is preferable to collect blood at this period and isolate MSCs therefrom.

The agent for osteogenesis according to the present invention contains as an active ingredient the MSCs which have been mobilized from bone marrow into blood by administration of Substance-P, and may further contain a bone substitute (scaffold). In one embodiment, the MSCs can be used in the state in which they are attached or loaded on the bone substitute. Any commercially available bone substitutes can be used for the present invention. Particularly, ceramic-based materials are preferable. One example of ceramic-based materials is hydroxy apatite tricalcium phosphate (HA-TCP).

Specifically, Substance-P was intravenously injected into rabbits (5 nmole/kg) twice (once a day), and the whole blood was collected on the 3rd day. After removing red blood cells from the blood by percoll gradient centrifugation, the isolated monocytes were cultured for about 2 weeks. As the time for the culture takes longer, the number of MSCs increased but the initially co-existing lymphocytes were all removed. After 2 weeks, only MSCs remained. These cells were separated by the treatment with trypsin/EDTA and counted.

2×10⁶ cells of MSCs were mixed with 40 mg of HA-TCP in a tube and cultured at 37° C. for 1 hour. In order to mix the contents evenly for the 1-hour culture, the tube was mildly beaten or shaken once every 10 minutes to help even attachment of the cells to the surface of HA-TCP.

A nude mouse was used as an animal model, and the MSCs-attached HA-TCP was subcutaneously transplanted on the back of the mouse. The timing of collecting transplant sample (i.e., the time to observe whether or not osteogenesis has occurred) is 8 or more weeks after transplantation, preferably 10 or more weeks after trans-plantation. The transplant was isolated from the mouse back and fixed with 4% paraformaldehyde for 3 days. After fixing, the transplant was put in 0.25 M EDTA for 3 weeks to remove calcium, and EDTA was changed three times a week. After removing calcium, the tissues were prepared as a paraffin block and cut longitudinally into 4 μm sections. After sufficient drying, Hematoxylin and Eosin (H&E) staining was performed to observe the histological morphology.

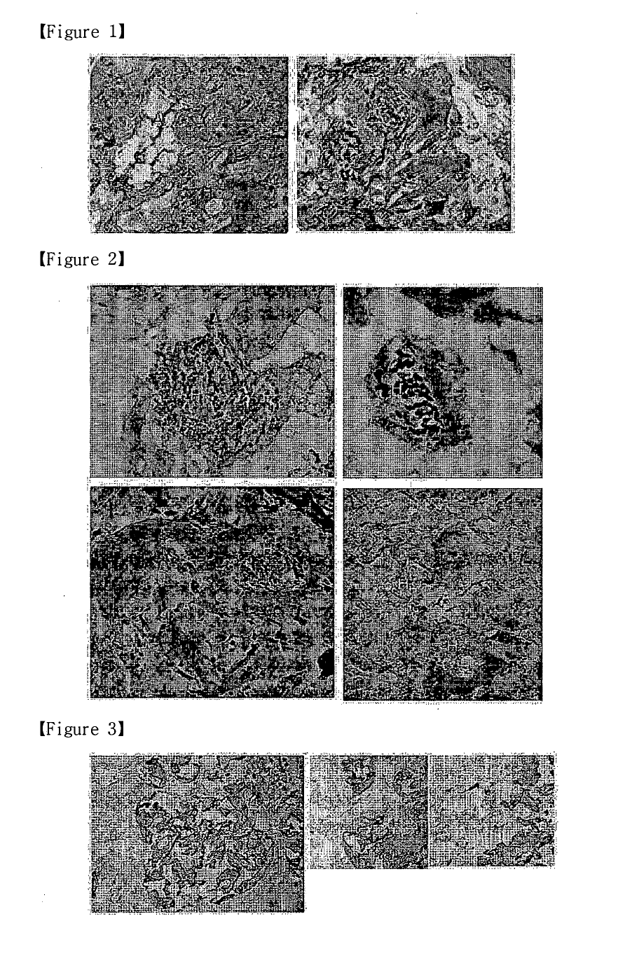

The result is shown in FIGS. 1 and 2. Specifically, FIG. 1 is a picture of the isolated transplant showing osteogenesis (pink color: collagen [bone matrix], A: fat tissues, H: bone marrow tissues, dark purple color: fibrous tissues). FIG. 2 is a high-resolution picture of FIG. 1. In view of the large formation of collagen of pink color, it was understood that osteogenesis substantially occurred.

Subsequently, the present inventors examined whether or not the bone had been generated from cells that are originated from rabbit. If bone matrix is formed by the transplanted cells, it must respond to rabbit antibodies. Other tissues such as fat tissues, bone marrow tissues and fibrous tissues should not respond. In order to confirm this, the isolated transplant was treated with antibodies that respond to rabbit collagen, and staining was performed. The result is shown in FIG. 3. As shown in FIG. 3, it was observed that only bone matrix was stained with these antibodies, and the surrounding tissues were not.

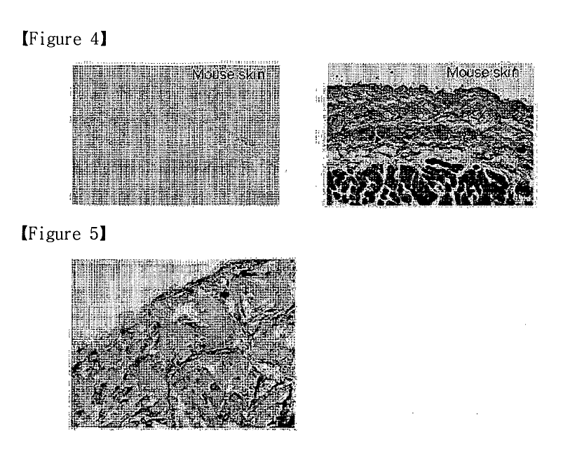

In addition, the same antibodies were treated with normal skin tissues of the mouse, and staining was performed. The result is shown in FIG. 4. FIG. 4 supports the fact that collagen exists in normal skin tissues of the mouse (see right picture), but such mouse collagen does not respond to the antibodies which respond to rabbit collagen (see left picture) (blue: collagen, red: cytoplasm).

From this result, it was understood that the antibodies used for the staining in FIG. 3 specifically respond only to rabbit. In addition, it was again confirmed that the bone matrix of the isolated transplant has been generated from the transplanted cells.

Furthermore, in order to examine whether or not the MSCs which have been mobilized from bone marrow into blood by administration of Substance-P according to the present invention would exhibit the capacity for osteogenesis in a degree equivalent to general MSCs that have been isolated directly from bone marrow, the present inventors isolated MSCs directly from rabbit bone marrow and performed the same experiment as above.

Specifically, MSCs were isolated from the tibia of a rabbit by bone marrow irrigation and aspiration, and cultured in mesenchymal stem cell growth medium (MSCGM). In the same manner as above, after mixing the cells with HA-TCP, they were subcutaneously transplanted on the back of a nude mouse. Ten weeks after transplantation, the transplant was isolated, and Hematoxylin and Eosin (H&E) staining was performed to observe the histological morphology. The result is shown in FIG. 5 (pink color: collagen [bone matrix], light purple color: residual HA-TCP).

From the comparison of FIG. 1 with FIG. 5, it was understood that the MSCs which have been mobilized from bone marrow into blood by administration of Substance-P exhibit the capacity for osteogenesis in a degree equivalent to general MSCs that have been isolated directly from bone marrow.

Based on the foregoing, the MSCs which have been mobilized from bone marrow into blood by administration of Substance-P can be used as an active ingredient for an agent for osteogenesis. For the agent for osteogenesis according to the present invention, the effective dosage of the MSCs is 2×10⁵ to 2×10⁷ cells, preferably 1×10⁶ to 3×10⁶ cells, most preferably 2×10⁶ cells per 40 mg of a bone substitute. However, these dosages can be increased or decreased depending on the size and degree of bone disease in patients, and on weight, age or sex of patients.

The agent for osteogenesis according to the present invention can be used for various kinds of fracture, bone necrosis disease or bone repair. In one embodiment, the agent for osteogenesis according to the present invention can be transplanted under the skin of a patient to the same degree as the target bone disease to be repaired. Eight weeks, preferably 10 weeks, after transplantation, the transplant can be isolated and inserted into the site of the target bone disease of the patient; thereby the target bone disease can be effectively treated.

As explained above, the agent for osteogenesis containing as an active ingredient the

MSCs which have been mobilized from bone marrow into blood by administration of Substance-P and the process of preparing the same according to the present invention can easily and conveniently isolate MSCs of bone marrow without causing pain to a patient, in contrast to the conventional methods of isolating MSCs directly from bone marrow of a patient. In addition, since the agent exhibits osteogenesis in a degree equivalent to general MSCs that have been isolated directly from bone marrow, the present invention for the first time proved that osteogenesis or bone repair is possible by cells isolated from blood.

Hereinafter, the present invention will be described in more detail with reference to the following working examples. The working examples are provided only to help understanding of the invention but are not to be construed as limiting the scope of the invention.

### EXAMPLES

### Example 1

### Isolation of MSCs from Blood after Administration of Substance-P and Their Culture

Substance-P (Calbiochem) was intravenously injected into a 1 month old rabbit (5 nmole/kg) twice (once a day), and the whole blood was collected on the 3rd day. After removing red blood cells from the blood by percoll gradient centrifugation, the isolated monocytes were cultured for 2 weeks. As the time for the culture takes longer, the number of MSCs increased but the initially co-existing lymphocytes were all removed. After 2 weeks, only MSCs remained. These cells were separated by the treatment with trypsin/EDTA and counted.

2×10⁶ cells of MSCs were mixed with 40 mg of HA-TCP in a tube and cultured at 37° C. for 1 hour. In order to mix the contents evenly for the 1-hour culture, the tube was mildly beaten or shaken once every 10 minutes to help even attachment of the cells to the surface of HA-TCP.

### Example 2

### Confirmation of the Capacity for Osteogenesis

A 6 weeks old female nude mouse was adapted to the environment under a breed condition at a temperature of 22±2° C. and relative humidity of 40-60% with supply of standard feed and water for one week, and then used in the experiment. The MSCs-attached HA-TCP prepared in Example 1 was subcutaneously transplanted on the back of the mouse. Ten weeks after transplantation, the transplant was isolated from the mouse back to observe whether or not osteogenesis occurred. The isolated transplant was fixed with 4% paraformaldehyde for 3 days. After fixing, the transplant was put in 0.25 M EDTA for 3 weeks to remove calcium, and EDTA was changed three times a week. After removing calcium, the tissues were prepared as a paraffin block and cut longitudinally into 4-gm sections. After sufficient drying, Hematoxylin and Eosin (H&E) staining was performed to observe the histological morphology.

The result is shown in FIGS. 1 and 2. Specifically, FIG. 1 is a picture of the isolated transplant showing osteogenesis (pink color: collagen [bone matrix], A: fat tissues, H: bone marrow tissues, dark purple color: fibrous tissues). FIG. 2 is a high-resolution picture of FIG. 1. In view of the large formation of collagen of pink color, it was understood that osteogenesis substantially occurred.

Subsequently, it was examined whether or not the bone had been generated from cells that were originated from rabbit. If bone matrix is formed by the transplanted cells, it must respond to rabbit antibodies. Other tissues such as fat tissues, bone marrow tissues and fibrous tissues should not respond. In order to confirm this, the isolated transplant was treated with antibodies that respond to rabbit collagen, and staining was performed. The result is shown in FIG. 3. As shown in FIG. 3, it was observed that only bone matrix was stained with these antibodies, and the surrounding tissues were not.

In addition, the same antibodies were treated with normal skin tissues of the mouse, and staining was performed. The result is shown in FIG. 4. FIG. 4 supports the fact that collagen exists in normal skin tissues of the mouse (see right picture), but such mouse collagen does not respond to the antibodies which respond to rabbit collagen (see left picture) (blue: collagen, red: cytoplasm).

From this result, it was understood that the antibodies used for the staining in FIG. 3 specifically respond to only rabbit. In addition, it was again confirmed that the bone matrix of the isolated transplant has been generated from the transplanted cells.

### Example 3

### Confirmation of the Capacity for Osteogensis of MSCs that have been Isolated Directly from Bone Marrow

MSCs were isolated from the tibia of a 1 month old rabbit by bone marrow irrigation and aspiration, and cultured in mesenchymal stem cell growth medium (MSCGM). In the same manner as Examples 1 and 2, after mixing the cells with HA-TCP, they were subcutaneously transplanted on the back of a nude mouse. Ten weeks after transplantation, the transplant was isolated, and Hematoxylin and Eosin (H&E) staining was performed to observe the histological morphology. The result is shown in FIG. 5 (pink color: collagen [bone matrix], light purple color: residual HA-TCP).

From the comparison of FIG. 1 with FIG. 5, it was understood that the MSCs which have been mobilized from bone marrow into blood by administration of Substance-P exhibit the capacity for osteogenesis in a degree equivalent to general MSCs that have been isolated directly from bone marrow.

1. An agent for osteogenesis containing as an active ingredient mesenchymal stem cells (MSCs) that have been mobilized from bone marrow into blood by administration of Substance-P.

2. The agent of claim 1, wherein the administration is by intravenous injection.

3. The agent of claim 1, wherein the MSCs are those which have been isolated from blood collected on the 3rd day after administration of Substance-P.

4. A process of preparing an agent for osteogenesis, comprising isolating MSCs that have been mobilized from bone marrow into blood by administration of Substance-P.

5. The process of claim 4, wherein the administration is by intravenous injection.

6. The process of claim 4, wherein the MSCs are isolated from blood collected on the 3rd day after administration of Substance-P.
